SEQUENCE LISTING (38 sequences)

SEQ ID NO: 1 - protein, 7 aa, synthetic construct
PAERPGV

SEQ ID NO: 2 - protein, 7 aa, synthetic construct
DGAFGSL

SEQ ID NO: 3 - protein, 7 aa, synthetic construct
DNNSKQN

SEQ ID NO: 4 - protein, 7 aa, synthetic construct
GNASKQE

SEQ ID NO: 5 - protein, 7 aa, synthetic construct
ATLGVST

SEQ ID NO: 6 - DNA, 21 bp, synthetic construct
  source 1..21: mol_type = other DNA
ccggctgagaggccgggggtg

SEQ ID NO: 7 - DNA, 21 bp, synthetic construct
  source 1..21: mol_type = other DNA
gatggggcgtttgggtctctg

SEQ ID NO: 8 - DNA, 21 bp, synthetic construct
  source 1..21: mol_type = other DNA
gataataattctaagcagaat

SEQ ID NO: 9 - DNA, 21 bp, synthetic construct
  source 1..21: mol_type = other DNA
ggtaatgcgtcgaagcaggag

SEQ ID NO: 10 - DNA, 21 bp, synthetic construct
  source 1..21: mol_type = other DNA
gctactctgggtgtgtcgact

SEQ ID NO: 11 - protein, 743 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSPAERPGVTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 12 - protein, 743 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSDGAFGSLTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 13 - protein, 743 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSDNNSKQNTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 14 - protein, 743 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSGNASKQETDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 15 - protein, 743 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSATLGVSTTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 16 - DNA, 2232 bp, synthetic construct
  source 1..2232: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcccggctgagaggccgggggtgacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 17 - DNA, 2232 bp, synthetic construct
  source 1..2232: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcgatggggcgtttgggtctctgacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 18 - DNA, 2232 bp, synthetic construct
  source 1..2232: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcgataataattctaagcagaatacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 19 - DNA, 2232 bp, synthetic construct
  source 1..2232: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcggtaatgcgtcgaagcaggagacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 20 - DNA, 2232 bp, synthetic construct
  source 1..2232: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcgctactctgggtgtgtcgactacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 21 - DNA, 41 bp, synthetic construct
  source 1..41: mol_type = other DNA
ccggctgagaggccgggggtgacagaccctgcgaccggaga

SEQ ID NO: 22 - DNA, 41 bp, synthetic construct
  source 1..41: mol_type = other DNA
cacccccggcctctcagccgggctgctgctctggagattga

SEQ ID NO: 23 - DNA, 41 bp, synthetic construct
  source 1..41: mol_type = other DNA
gatggggcgtttgggtctctgacagaccctgcgaccggaga

SEQ ID NO: 24 - DNA, 41 bp, synthetic construct
  source 1..41: mol_type = other DNA
cagagacccaaacgccccatcgctgctgctctggagattga

SEQ ID NO: 25 - DNA, 41 bp, synthetic construct
  source 1..41: mol_type = other DNA
gataataattctaagcagaatacagaccctgcgaccggaga

SEQ ID NO: 26 - DNA, 41 bp, synthetic construct
  source 1..41: mol_type = other DNA
attctgcttagaattattatcgctgctgctctggagattga

SEQ ID NO: 27 - DNA, 41 bp, synthetic construct
  source 1..41: mol_type = other DNA
ggtaatgcgtcgaagcaggagacagaccctgcgaccggaga

SEQ ID NO: 28 - DNA, 41 bp, synthetic construct
  source 1..41: mol_type = other DNA
ctcctgcttcgacgcattaccgctgctgctctggagattga

SEQ ID NO: 29 - DNA, 41 bp, synthetic construct
  source 1..41: mol_type = other DNA
gctactctgggtgtgtcgactacagaccctgcgaccggaga

SEQ ID NO: 30 - DNA, 41 bp, synthetic construct
  source 1..41: mol_type = other DNA
agtcgacacacccagagtagcgctgctgctctggagattga

SEQ ID NO: 31 - DNA, 37 bp, synthetic construct
  source 1..37: mol_type = other DNA
gcatctttgaacaataaatgatttaaatcaggtatgg

SEQ ID NO: 32 - DNA, 46 bp, synthetic construct
  source 1..46: mol_type = other DNA
gttcaactgaaacgaatcaatttattgattaacaggcaattacagg

SEQ ID NO: 33 - DNA, 19 bp, synthetic construct
  source 1..19: mol_type = other DNA
gctggagtacaactacaac

SEQ ID NO: 34 - DNA, 18 bp, synthetic construct
  source 1..18: mol_type = other DNA
tggcggatcttgaagttc

SEQ ID NO: 35 - DNA, 19 bp, synthetic construct
  source 1..19: mol_type = other DNA
ctgggctacactgagcacc

SEQ ID NO: 36 - DNA, 21 bp, synthetic construct
  source 1..21: mol_type = other DNA
aagtggtcgttgagggcaatg

SEQ ID NO: 37 - DNA, 75 bp, synthetic construct
  source 1..75: mol_type = other DNA
gggactgtggcagtcaatctccagagcagcagcnnknnknnknnknnknnknnkacagaccctgcgaccggagat

SEQ ID NO: 38 - DNA, 45 bp, synthetic construct
  source 1..45: mol_type = other DNA
cggtttattgattaacaatcgattacaggggacgggtgaggtaac

(19) United States
(12) Patent Application Publication
(10) Pub. No.: US 2025/0297284 A1
(43) Pub. Date: Sep. 25, 2025

(54) MUTANT OF ADENO-ASSOCIATED VIRUS AND USE THEREOF

(71) Applicant: PACKGENE BIOTECH CO., LTD., Guangzhou (CN)

(72) Inventors: Ying Fan, Guangzhou (CN); Huapeng Li, Guangzhou (CN); Ye Bu, Guangzhou (CN); Yujian Zhong, Guangzhou (CN); Zhiyong Dai, Guangzhou (CN); Keqin Tan, Guangzhou (CN); Youwei Zhang, Guangzhou (CN)

(21) Appl. No.: 19/231,418

(22) Filed: Jun. 6, 2025

Related U.S. Application Data
(63) Continuation of application No. PCT/CN2023/074196, filed on Feb. 2, 2023.

(30) Foreign Application Priority Data
Dec. 8, 2022 (CN) 202211576110.3

(51) Int. Cl. C12N 15/86; C07K 14/005

(52) U.S. Cl. CPC C12N 15/86; C07K 14/005; C12N 2750/14122; C12N 2750/14143

(57) ABSTRACT

Provided are a mutant of an adeno-associated virus (AAV) targeting a resting or activated T cell, a use of the mutant of the AAV, and a heterologous peptide targeting a T cell. An amino acid sequence of the heterologous peptide is set forth in any one of SEQ ID NOS: 1-5. The mutant of the AAV exhibits high efficiency in targeting resting or activated T cells, and has advantages such as low dose, strong infectivity, and high safety. A recombinant adeno-associated virus (rAAV) can be constructed from a mutant of an AAV capsid protein of the present application. Nucleic acid sequences carried by rAAV can not be integrated into the genome of the host cell. T he rAAV can be used for the quick infection and reinfusion of activated T cells, which reduces the unnecessary quality control and in vitro dwell time.

### CROSS-REFERENCE TO RELATED APPLICATIONS

The present application is a Continuation Application of PCT application No. PCT/CN2023/074196 filed on Feb. 2, 2023, which claims the benefit of Chinese Patent Application No. 202211576110.3 filed on Dec. 8, 2022. The contents of the above-identified applications are hereby incorporated by reference.

### REFERENCE TO SEQUENCE LISTING

This application includes a Sequence Listing filed electronically as an XML file named “U.S. Pat. No. 2,501,117H-PCT_SL.xml”, created on Jun. 5, 2025, with a size of 49,435 bytes. The Sequence Listing is incorporated herein by reference.

### TECHNICAL FIELD

The present application relates to the technical field of biomedicine, and specifically relates to a mutant of an adeno-associated virus (AAV) and a use thereof.

### BACKGROUND

AAV is a non-pathogenic and replication-defective virus. The genome of AAV is a single-stranded DNA fragment with a length of about 4.7 kb. The genome of AAV is encapsulated within a non-enveloped viral capsid, and can be divided into the following three functional regions: two open reading frames and an inverted terminal repeat. Recombinant adeno-associated virus (rAAV) vectors are derived from non-pathogenic wild-type AAVs. rAAV vectors are a class of major gene vectors. rAAV vectors are widely used in the fields of gene function research and gene therapy due to advantages such as broad host spectrum, non-pathogenicity, low immunogenicity, long-term stable expression of exogenous genes, prominent diffusion performance, and stable physical properties. Different viral serotypes exhibit distinct tropisms for different tissue and cell types, resulting in varying transfection efficiencies.

Tumor immunotherapy relies on the specific abilities of an immune system itself to recognize and kill tumor cells. Tumor immunotherapy is currently one of the most promising therapeutic approaches in the tumor treatment field. In recent years, with the in-depth research on tumor immunotherapy, various types of therapies have exhibited excellent efficacy in treating refractory and relapsed tumors, such as antibody-drug conjugates, bispecific antibodies, CAR-T therapy, and TCR-T therapy. T cells play a crucial role in destroying diseased cells throughout the body. Studies on immune checkpoint inhibitors and tumor infiltrating lymphocytes have demonstrated the potential of T cells in cancer treatments. However, T cells require appropriate tumor specificity, a sufficient quantity, and an ability of overcoming any local immunosuppressive factors to exert a therapeutic effect. Therefore, the development of a viral vector that can efficiently target T cells without compromising the activity of T cells holds significant clinical benefits and commercial significance.

### SUMMARY

An objective of the present application is to overcome the shortcomings of the prior art and provide an AAV mutant targeting a resting or activated T cell, and a use of the AAV mutant. The AAV mutant shows enhanced infectivity for resting or activated T cells, and has advantages such as low dose, strong infectivity, and high safety.

In order to achieve the above objective, the present application adopts the following technical solutions:

In a first aspect, the present application provides a heterologous peptide targeting a T cell, where an amino acid sequence of the heterologous peptide is set forth in any one of SEQ ID NOS: 1-5.

The present application also provides a mutant of an AAV capsid protein including the heterologous peptide. A rAAV mutant (or vector) constructed accordingly can efficiently target resting or activated T cells, and has advantages such as low dose, strong infectivity, and high safety.

As a preferred embodiment of the heterologous peptide in the present application, a nucleotide sequence encoding the heterologous peptide is set forth in any one of SEQ ID NOS: 6-10.

In a second aspect, the present application provides a mutant of an AAV capsid protein targeting a T cell, including the heterologous peptide.

rAAV can be constructed from the mutant of the AAV capsid protein of the present application without being integrated into a genome. The rAAV can be used for the quick infection and reinfusion of activated T cells, which reduces the unnecessary quality control and in vitro dwell time. The rAAV mutant constructed from the mutant of the AAV capsid protein of the present application also exhibits very excellent infectivity for T cells stimulated with a stimulating factor. The infection of resting or activated T cells with the rAAV mutant carrying the mutant of the AAV capsid protein of the present application has great clinical values and commercial application prospects.

As a preferred embodiment of the mutant of the AAV capsid protein in the present application, the mutant of the AAV capsid protein is produced by inserting the heterologous peptide into an AAV capsid protein or substituting 5 to 20 amino acids of the AAV capsid protein with the heterologous peptide.

As a preferred embodiment of the mutant of the AAV capsid protein in the present application, an insertion site for the heterologous peptide is located between amino acids 588 and 589 of the AAV capsid protein. The amino acid 588 refers to the 588th amino acid in the amino acid sequence of the AAV capsid protein.

As a preferred embodiment of the mutant of the AAV capsid protein in the present application, an amino acid sequence of the mutant of the AAV capsid protein is set forth in any one of SEQ ID NOS: 11-15.

As a preferred embodiment of the mutant of the AAV capsid protein in the present application, a nucleotide sequence encoding the mutant of the AAV capsid protein is set forth in any one of SEQ ID NOS: 16-20.

In a third aspect, the present application provides a rAAV targeting a T cell, including the mutant of the AAV capsid protein.

The rAAV mutant in the present application exhibits improved infectivity for resting or activated T cells, and has advantages such as low dose, strong infectivity, and high safety. In particular, the infection of inactivated T cells can allow the collection, infection, and reinfusion of T cells on the same day, which can greatly reduce the preparation cost and minimize the influence on the activity of T cells.

As a preferred embodiment of the rAAV in the present application, the rAAV further includes a heterologous target gene.

As a preferred embodiment of the rAAV in the present application, the heterologous target gene encodes any gene product selected from the group consisting of interference RNA, an aptamer, an endonuclease, and a guide RNA.

In a fourth aspect, the present application provides a pharmaceutical composition for delivering a gene product to a cell of a subject, including the heterologous peptide, the mutant of the AAV capsid protein, or the rAAV.

As a preferred embodiment of the pharmaceutical composition in the present application, the cell is an immune cell.

In a fifth aspect, the present application provides a method for infecting a resting or activated T cell, including allowing the rAAV to contact the resting or activated T cell.

In a sixth aspect, the present application provides a pharmaceutical composition for tumor immunotherapy, including the heterologous peptide, the mutant of the AAV capsid protein, or a therapeutically effective amount of the rAAV.

As a preferred embodiment of the pharmaceutical composition for tumor immunotherapy in the present application, the tumor immunotherapy includes a CAR-T therapy or a TCR-T therapy.

Compared with the prior art, the present application has the following beneficial effects:

The rAAV mutant constructed from the mutant of the AAV capsid protein in the present application can efficiently target T cells, and has advantages such as low dose, strong infectivity, and high safety. rAAV can be constructed from the mutant of the AAV capsid protein of the present application without being integrated into a genome. The rAAV can be used for the quick infection and reinfusion of activated T cells, which reduces the unnecessary quality control and in vitro dwell time. The rAAV mutant constructed from the mutant of the AAV capsid protein of the present application also exhibits very excellent infectivity for T cells stimulated with a stimulating factor. The infection of resting or activated T cells with the rAAV carrying the mutant of the AAV capsid protein of the present application has great clinical values and commercial application prospects.

### BRIEF DESCRIPTION OF THE DRAWINGS

FIG. 1A to FIG. 1F show fluorescence intensities of enhanced green fluorescent proteins (eGFPs) in resting T cells infected with different rAAVs (at 72 h after infection) that are detected by fluorescence microscopy, where FIG. 1A shows rAAV6, FIG. 1B to FIG. 1F show viruses 1 to 5 including mutants of the AAV6 capsid protein (namely, AAV6 mutants 1 to 5), respectively, and the bright field refers to bright-field imaging;

FIG. 2A to FIG. 2F show fluorescence intensities of eGFPs in resting T cells infected with different rAAVs (at 96 h after infection) that are detected by fluorescence microscopy, where FIG. 2A shows rAAV6, FIG. 2B to FIG. 2F show viruses 1 to 5 including mutants of the AAV6 capsid protein (namely, AAV6 mutants 1 to 5), respectively, and the bright field refers to bright-field imaging;

FIG. 3 shows relative intensities of eGFP mRNA in resting T cells infected with viruses 1 to 5 containing the mutants of the AAV6 capsid protein (namely, AAV6 mutants 1 to 5) for 72 h that are detected by reverse transcription-quantitative polymerase chain reaction (RT-qPCR);

FIG. 4A to FIG. 4I show percentages and intensities of eGFP-positive cells among resting T cells infected with different rAAVs for 96 h that are detected by flow cytometry, where

FIG. 4A shows a blank control, FIG. 4B and FIG. 4C show control rAAV6, FIG. 4D to FIG. 4H show viruses 1 to 5 including mutants of the AAV6 capsid protein (namely, AAV6 mutants 1 to 5), respectively, and FIG. 4I is a bar chart of statistical results of proportions of eGFP-positive cells in groups;

FIG. 5A and FIG. 5B show infection abilities of different rAAVs for resting T cells that are detected through luciferase activity assay, where FIG. 5A shows luciferase activities determined at 72 h after infection and FIG. 5B shows luciferase activities determined at 96 h after infection;

FIG. 6 shows fluorescence intensities of eGFPs in resting T cells infected with control rAAV6 and virus 1 containing a mutant of the AAV6 capsid protein (namely, AAV6 mutant 1) at different multiplicity of infection (MOI) values for 72 h that are detected by fluorescence microscopy, where the bright field refers to bright-field imaging;

FIG. 7 shows relative intensities of eGFP mRNA in resting T cells infected with control rAAV6 and virus 1 containing a mutant of the AAV6 capsid protein (namely, AAV6 mutant 1) at different MOI values for 72 h that are detected by RT-qPCR;

FIG. 8A to FIG. 8F show fluorescence intensities of eGFPs in activated T cells infected with different rAAVs (at 72 h after infection) that are detected by fluorescence microscopy, where FIG. 8A shows rAAV6, and FIG. 8B to FIG. 8F show viruses 1 to 5 including mutants of the AAV6 capsid protein (namely, AAV6 mutants 1 to 5), respectively;

FIG. 9A to FIG. 9E show percentages and intensities of eGFP-positive cells among activated T cells infected with different rAAVs at various MOI values for 96 h that are detected by flow cytometry, where FIG. 8A shows control rAAV6, FIG. 8B to FIG. 8D show viruses 1 to 3 including mutants of the AAV6 capsid protein (namely, AAV6 mutants 1 to 3), respectively, and FIG. 8E is a bar chart of statistical results of proportions of eGFP-positive cells in groups; and

FIG. 10 shows relative intensities of eGFP mRNA in activated T cells infected with different rAAVs at various MOI values for 96 h that are detected by RT-qPCR.

### DETAILED DESCRIPTION

To well explain the objective, technical solutions, and advantages of the present application, the present application will be further explained below with reference to specific embodiments. It should be understood by those skilled in the art that the specific embodiments described herein are merely intended to explain the present application, rather than to limit the present application.

In the following embodiments, unless otherwise specified, the experimental methods adopted are conventional, and the materials and reagents adopted are commercially available. A GenBank accession number for AAV6 VP1 is AF028704.1.

### Example 1 Selection of AAV6 Mutants for Effectively Infecting Inactivated T Cells

### (1) Construction of a Backbone Vector for an AAV6 Library

The backbone vector for the AAV6 library included a CAG promoter, an intron, a mutated AAV6 CAP sequence [a sequence after the amino acid N583 in an AAV6 CAP sequence was removed, and T of N583 (which was the base T in a codon AAT encoding a 583rd amino acid N) and a sequence of a front segment of polyA (namely, the first 5 bases “GTACA” of the polyA sequence) constituted a site BsrG I (TGTACA) for the subsequent enzyme cleavage of the backbone], and polyA. The above sequences were synthesized through gene synthesis, and inserted between inverted terminal repeats (ITRs) of a rAAV vector to produce the backbone vector for the AAV6 library.

### (2) Construction of a Mutated Rep-CAP Vector

A stop codon was introduced within the first 20 bp of each of start codons of VP1, VP2, and VP3 in a CAP sequence of AAV6, such that a Rep-CAP vector expressed a Rep protein, but did not express VP1, VP2, and VP3 proteins of CAP, which avoided the contamination to the CAP sequence of the parent AAV6. The above sequence was synthesized through gene synthesis and inserted into a Rep-CAP vector to replace a corresponding CAP sequence.

(3) Construction of a Vector Library Including Random 7-mer Peptides (Each Including 7 Amino Acids)

Two primers [an insertion site was located between S588 (the 588th amino acid S) and T589 (the 589th amino acid T) in an amino acid sequence for an AAV6 capsid protein, an upstream primer targeted a nucleotide sequence after T589 in a template (a CAP sequence), and a downstream primer targeted a terminal sequence of a CAP nucleotide sequence] were designed. 5′ termini of both the upstream and downstream primers had a consistent homologous arm sequence of 15 bp or more with the backbone. In addition, in the upstream primer, a 21 bp nucleic acid sequence (7 * NNK, where N represented A, T, C, or G, and K represented a keto base, namely, T or G) was introduced between the homologous arm sequence and a targeting sequence to introduce a nucleotide sequence encoding a random 7-mer peptide into a CAP nucleotide sequence.

Base sequences of the two primers (5′->3′) were as follows:

V6-7P-F:
(SEQ ID NO: 21)
GGGACTGTGGCAGTCAATCTCCAGAGCAGCAGCNNKNNKNNKNNKNNKN
NKNNKACAGACCCTGCGACCGGAGAT;
and
V6-Stop-R:
(SEQ ID NO: 22)
CGGTTTATTGATTAACAATCGATTACAGGGGACGGGTGAGGTAAC.

With a vector carrying the AAV6 CAP nucleotide sequence as a template, the above primers were used to conduct PCR amplification to produce a fragment carrying a random sequence. Gel electrophoresis and gel extraction were conducted to produce purified nucleic acid fragments for a random 7-mer peptide library. The nucleic acid fragments were ligated to the backbone vector for the AAV6 library (which had been purified through BsrG I enzyme cleavage and gel extraction) through Gibson assembly based on homologous recombination. A vector produced after the ligation was purified with a PCR product purification kit and then digested with Plasmid Safe DNase to remove the fragments not ligated. Finally, purification was conducted with a PCR product purification kit to produce the AAV6 vector library.

### (4) Construction of a Virus Library of AAV6 Mutants

The mutated Rep-Cap plasmid, the AAV6 vector library, and a pHelper plasmid were co-transfected into HEK-293T cells. AAV was purified through iodixanol-based gradient ultracentrifugation. When a viral titer was measured to be 1×10¹² GC/mL to 1×10¹³ GC/mL, a virus library of AAV6 mutants was obtained and stored at −80° C. for later use.

### (5) Screening of AAV6 Mutants in T Cells

### 1) Recovery and AAV Infection of T Cells

An RPMI 1640 medium was pre-warmed at 37° C. Frozen CD3⁺ T cells were taken and quickly thawed. Recovered cells were transferred to a 50 mL centrifuge tube, 15 mL of an RPMI 1640 medium including 1% of P/S and 10% of fetal bovine serum (FBS) was added to the centrifuge tube, and the centrifuge tube was centrifuged at 300 g for 10 min to 15 min. The cells were resuspended in 1 mL of an RPMI 1640 medium including 1% of P/S and 10% of FBS, and counted (staining was conducted with trypan blue, and a total number of cells and a number of dead cells were counted). 5×10⁵ cells were added to each well of a cell culture plate. The virus library of AAV6 mutants was added at a dose of 5×10⁸, 5×10⁹, or 5×10⁵ GC/well to infect the inactivated T cells. After 1 h or 3 h of the infection, rhIL-2 was added at a final concentration of 50 U/mL to each well, and thorough mixing was conducted through gentle pipetting. 2 h later, a T cell activator (i.e., anti-CD3/CD28 antibodies) was added at a final concentration of 25 μL/mL to each well, and thorough mixing was conducted through gentle pipetting. Culturing was allowed in an incubator for 48 h (37° C., 5% CO₂).

### 2) Total RNA Extraction and Reverse Transcription-Polymerase Chain Reaction (RT-PCR)

A cell suspension was pipetted into a 1.5 mL centrifuge tube and centrifuged at 300 g for 10 min to collect cells, and a resulting supernatant was discarded. RNA extraction was conducted according to the instructions of TransZol Up Plus RNA Kit (Beijing TransGen Biotech Co., Ltd., Item No.: ER501). Based on an extracted RNA sample, first-strand cDNA was synthesized with PrimeScript™ IV 1st strand cDNA Synthesis Mix (TAKARA, Item No.: 6215A). Two rounds of PCR amplification were conducted with NEB Q5. The first round of PCR amplification was conducted with outer primers. The second round of PCR amplification was conducted with a gel-extracted product from the first round of PCR amplification as a template or with NGS primers. A PCR product with a corresponding band size was recovered with a gel and sent to a company for next-generation sequencing (NGS). Alternatively, amplification and gel extraction were conducted with primers used for library construction, and then library construction of sub-vectors, virus packaging, screening, etc. were conducted as above. mutants 1 to 5 of the AAV capsid protein were selected. For VP1, amino acid sequences were set forth in SEQ ID NOS: 11-15, respectively, and nucleotide sequences were set forth in SEQ ID NOS: 16-20, respectively. For a targeting peptide in VP1, amino acid sequences were set forth in SEQ ID NOS: 1-5, respectively, and nucleotide sequences were set forth in SEQ ID NOS: 6-10, respectively.

### Example 2 Construction of Mutants of the AAV Capsid Protein and Production of Viruses

With rAAV6 as a control, mutants 1 to 5 of the AAV capsid protein were constructed, which was specifically as follows:

### (1) Construction of Mutant Serotype Vectors and Extraction of Plasmids

A Rep-CAP plasmid was subjected to double-enzyme cleavage with Smi I and BshT I. Gel electrophoresis was conducted, and a band of about 5,000 bp was cut and subjected to extraction to produce a cleaved backbone fragment. According to the Cap sequences of the selected target mutants 1 to 5, primers were designed to construct plasmids for the target mutants AAV. With the Rep-CAP plasmid for AAV6 as a template, PCR amplification was conducted using primers F1 and R1 to produce a target product 1. Similarly, with the Rep-CAP plasmid for AAV6 as a template, PCR amplification was conducted with primers F2 and R2 to produce a target product 2. There was a homologous arm sequence between the cleaved backbone fragment and the target product 1 or 2 and between the target products 1 and 2. Thus, a plurality of fragments could be assembled into a complete vector through Gibson assembly.

In the construction of a vector for mutant 1 of an AAV capsid protein, primers for a PCR product 1 were Cap-F and FF07-R, and primers for a PCR product 2 were FF07-F and Cap-R. The primer sequences (5′ to 3′) involved were as follows:

FF07-F:
(SEQ ID NO: 23)
CCGGCTGAGAGGCCGGGGGTGACAGACCCTGCGACCGGAGA;
and
FF07-R:
(SEQ ID NO: 24)
CACCCCCGGCCTCTCAGCCGGGCTGCTGCTCTGGAGATTGA.

In the construction of a vector for mutant 2 of an AAV capsid protein, primers for a PCR product 1 were Cap-F and FF09-R, and primers for a PCR product 2 were FF09-F and Cap-R. The primer sequences (5′ to 3′) involved were as follows:

FF09-F:
(SEQ ID NO: 25)
GATGGGGCGTTTGGGTCTCTGACAGACCCTGCGACCGGAGA;
and
FF09-R:
(SEQ ID NO: 26)
CAGAGACCCAAACGCCCCATCGCTGCTGCTCTGGAGATTGA.

In the construction of a vector for mutant 3 of an AAV capsid protein, primers for a PCR product 1 were Cap-F and FF10-R, and primers for a PCR product 2 were FF10-F and Cap-R. The primer sequences (5′ to 3′) involved were as follows:

FF10-F:
(SEQ ID NO: 27)
GATAATAATTCTAAGCAGAATACAGACCCTGCGACCGGAGA;
and
FF10-R:
(SEQ ID NO: 28)
ATTCTGCTTAGAATTATTATCGCTGCTGCTCTGGAGATTGA.

In the construction of a vector for mutant 4 of an AAV capsid protein, primers for a PCR product 1 were Cap-F and FF15-R, and primers for a PCR product 2 were FF15-F and Cap-R. The primer sequences (5′ to 3′) involved were as follows:

FF15-F:
(SEQ ID NO: 29)
GGTAATGCGTCGAAGCAGGAGACAGACCCTGCGACCGGAGA;
and
FF15-R:
(SEQ ID NO: 30)
CTCCTGCTTCGACGCATTACCGCTGCTGCTCTGGAGATTGA.

In the construction of a vector for mutant 5 of an AAV capsid protein, primers for a PCR product 1 were Cap-F and FF17-R, and primers for a PCR product 2 were FF17-F and Cap-R. The primer sequences (5′ to 3′) involved were as follows:

FF17-F:
(SEQ ID NO: 31)
GCTACTCTGGGTGTGTCGACTACAGACCCTGCGACCGGAGA;
and
FF17-R:
(SEQ ID NO: 32)
AGTCGACACACCCAGAGTAGCGCTGCTGCTCTGGAGATTGA.

Sequences of the primers Cap-F and Cap-R involved in the construction of the vectors for mutants 1 to 5 of the AAV capsid protein were as follows:

Cap-F:
(SEQ ID NO: 33)
GCATCTTTGAACAATAAATGATTTAAATCAGGTATGG;
and
Cap-R:
(SEQ ID NO: 34)
GTTCAACTGAAACGAATCAATTTATTGATTAACAGGCAATTACAGG.

A clean 200 μL PCR tube was taken, labeled properly, and placed in an ice box. A reaction system was prepared from the cleaved backbone fragment, the target fragment 1, and the target fragment 2, where a molar ratio of the cleaved backbone fragment to the target fragments was 1:3. The reaction system was subjected to recombination and ligation at 50° C. for 30 min in a PCR instrument. 50 μL of a competent cell suspension was taken and thawed on ice. 10 μL of a ligation product was mixed with DH5α competent cells, and incubation was allowed on ice for 20 min to 30 min. A heat shock was conducted at 42° C. for 45 s, and incubation was then immediately allowed in an ice bath for 2 min. Then 400 μL of a recovery SOC medium (without antibiotics) was added, and incubation was allowed at 37° C. and 200 rpm for 1 h. Resulting transformed cells were uniformly coated on an Amp-resistant plate (50 μg/mL) and cultured at 32° C. for 18 h. Single colonies were picked, inoculated into 4 mL of a liquid LB medium (with Amp resistance), and cultured at 32° C. for 18 h to allow expansion.

A resulting bacterial solution was centrifuged at 12,000 rpm for 1 min to produce a first supernatant, and the first supernatant was discarded. 250 μL of a buffer P1/RNaseA mixture was added, and high-speed vortexing was conducted to resuspend bacteria. 250 μL of a buffer P2 was added, and thorough mixing was allowed by inverting up and down 8 times to 10 times. 350 μL of a buffer P3 was added, and thorough mixing was allowed by immediately inverting up and down 8 times to 10 times to make a solution completely neutralized. Centrifugation was conducted at 13,000 rpm for 10 min to produce a second supernatant, and the second supernatant was collected and loaded on a column. Centrifugation was conducted at 12,000 rpm for 1 min to produce a third supernatant, and the third supernatant was discarded. 500 μL of PW1 was added, centrifugation was conducted at 12,000 rpm for 1 min to produce a fourth supernatant, and the fourth supernatant was discarded. 600 μL of PW2 was added, centrifugation was conducted at 12,000 rpm for 1 min to produce a fifth supernatant, and the fifth supernatant was discarded. 600 μL of PW2 was added, centrifugation was conducted at 12,000 rpm for 1 min to produce a sixth supernatant, and the sixth supernatant was discarded. Idling centrifugation was conducted at 12,000 rpm for 2 min. 30 μL to 50 μL of an eluent preheated at 55° C. was added, and standing was allowed for 2 min. Then centrifugation was conducted at 12,000 rpm for 1 min. A concentration was detected by a microvolume nucleic acid quantification instrument.

Resulting plasmids each were tested for a concentration. 10 μL of each positive plasmid identified by enzyme cleavage was taken and sent for sequencing. Positive plasmids were stored at −20° C. Sequencing results showed that the positive plasmids could encode mutants of the capsid protein VP1. Finally, based on a viral load required in the later test, the relevant Helper plasmid, the Rep-Cap plasmid in each group (AAV6 and AAV6 mutants 1 to 4), and the GOI plasmid (including scAAV, CAG, EGFP, WPREs, and SV40pA) were extracted. (2) Packaging and purification of mutant virus serotypes

The Rep-Cap plasmid in each group (wild-type AAV6 and mutants of the AAV6 capsid protein), a plasmid expressing eGFP or a plasmid expressing firefly luciferase, and a pHelper plasmid were co-transfected into HEK-293T cells at appropriate amounts. AAV was purified through iodixanol-based gradient ultracentrifugation. When a viral titer was measured to be 1×10¹² GC/mL to 1×10¹³ GC/mL, control wild-type AAV6 and viruses 1 to 5 containing mutants of the AAV6 capsid protein were obtained and stored at −80° C. for later use.

### Example 3 Contrast Detection of Various Indexes for Resting T Cells Infected with Mutant Serotypes

### (1) AAV Infection and Culturing of T Cells

Groups: Control AAV6 and viruses 1 to 5 containing mutants of the AAV6 capsid protein

An RPMI 1640 medium was pre-warmed at 37° C. Frozen CD3⁺ T cells were taken and rapidly thawed. Recovered cells were transferred to a 50 mL centrifuge tube, 15 mL of an RPMI 1640 medium including 1% of P/S and 10% of FBS was added to the centrifuge tube, and the centrifuge tube was centrifuged at 300 g for 10 min to 15 min. Cells were resuspended with 1 mL of an RPMI 1640 medium including 1% of P/S and 10% of FBS, and counted (staining was conducted with trypan blue, and a total number of cells and a number of dead cells were counted). According to cell counting results, a cell density was adjusted to 1×10⁶ cells/mL. According to the experimental grouping, 500 μL of a cell suspension (5×10⁵ cells/well) was added to a 24-well cell culture plate, or 100 μL of a cell suspension (1×10⁵ cells/well) was added to a 96-well cell culture plate. T cells in each group were infected at MOI of 1E4 (MOI for a control group of high-dose AAV6 was 1E5). In an experiment to compare viral infectivity at different MOIs, T cells in each group were infected at MOI of 1E2, 1E3, or 1E4. At 4 h after infection, rhIL-2 was added at a final concentration of 50 U/mL to each well, and thorough mixing was conducted through gentle pipetting. 2 h later, a T cell activator (i.e., anti-CD3/CD28 antibodies) was added at a final concentration of 25 μL/mL to each well, and thorough mixing was conducted through gentle pipetting. Culturing was allowed in an incubator (37° C., 5% CO₂).

### (2) Fluorescence Observation

At 72 h and 96 h, T cells in each group were subjected to fluorescence imaging by fluorescent microscopy (with consistent imaging parameters and exposure times).

(3) Detection of Expression Levels of Target mRNAs

### 1) Total RNA Extraction:

A cell suspension in each group was pipetted to a 1.5 mL centrifuge tube and centrifuged at 300 g for 15 min to collect cells, and a resulting supernatant was discarded. RNA extraction was conducted according to the instructions of TransZol Up Plus RNA Kit (Beijing TransGen Biotech Co., Ltd., Item No.: ER501). 300 μL of TranZol up was added to cells in each tube, and then 60 μL of chloroform was added. Shaking was conducted vigorously for 30 s, and incubation was conducted at room temperature for 3 min. Centrifugation was conducted at 12,000 g and 4° C. for 10 min. A resulting colorless aqueous phase was transferred to a new 1.5 mL RNase-free EP tube, an equal volume of absolute ethanol was added, and thorough mixing was allowed through gentle inverting to produce a solid-liquid mixture. The solid-liquid mixture was loaded on a spin column and centrifuged at 12,000 g and room temperature for 30 s to produce a first filtrate, and the first filtrate was discarded. 500 μL of CB9 was added, centrifugation was conducted at 12,000 g and room temperature for 30 s to produce a second filtrate, and the second filtrate was discarded, which was repeated once. 500 μL of WB9 was added, centrifugation was conducted at 12,000 g and room temperature for 30 s to produce a third filtrate, and the third filtrate was discarded, which was repeated once. Centrifugation was conducted at 12,000 g and room temperature for 2 min to completely remove the residual ethanol. The spin column was placed in a 1.5 mL RNase-free EP tube, 50 μL of RNase-free water was added to a center of the spin column, and standing was allowed at room temperature for 1 min. Centrifugation was conducted at 12,000 g and room temperature for 1 min to elute RNA. A concentration of RNA in a sample was detected by a microvolume nucleic acid quantification instrument. OD260/280 and OD260/230 were determined. The extracted RNA was stored at −80° C. Based on an RNA sample in each group, first-strand cDNA was synthesized with HiScript® III RT SuperMix for qPCR (+gDNA wiper) (Nanjing Vazyme Biotech Co., Ltd., Item No.: R323).

2) Quantitative Polymerase Chain Reaction (qPCR) Experiment

With cDNA in each group as a template, a qPCR system was prepared according to the instructions of 2× SYBR Green qPCR Master Mix (BIMAKE, Item No.: B212203), as shown in Table 1:

TABLE 1
qPCR system

| Reagent | Volume | |
|---|---|---|
| 2× SYBR Green qPCR | 10 | μL |
| Master Mix | | |
| Template | 1 | μL |
| Upstream primer | 0.5 | μL |
| Downstream primer | 0.5 | μL |
| ROX Reference Dye | 0.4 | μL |

| | |
|---|---|
| Deionized water | Up to 20 μL |

Primer sequences (5′->3′) were as follows:

EGFP-Tf:
(SEQ ID NO: 35)
GCTGGAGTACAACTACAAC;
EGFP-Tr:
(SEQ ID NO: 36)
TGGCGGATCTTGAAGTTC;
GAPDH101-F:
(SEQ ID NO: 37)
CTGGGCTACACTGAGCACC;
and
GAPDH101-R:
(SEQ ID NO: 38)
AAGTGGTCGTTGAGGGCAATG.

A qPCR program was set as in Table 2:

TABLE 2
qPCR program

| | 1 | 2 | | | | | | 3 | | | | | |
|---|---|---|---|---|---|---|---|---|---|---|---|---|---|
| Step | Hot-Start DNA Polymerase | PCR | | | | | | Melt Curve | | | | | |
| | Activation | | | | | | | | | | | | |
| | Hold | Cycle (40 cycles) | | | | | | Cycle (1 cycle) | | | | | |
| | | Denature | | Anneal | | Extend | | | | | | | |
| Temp | 95° C. | 95° | C. | 60° | C. | 72° | C. | 95° | C. | 60° | C. | 95° | C. |
| Time | 30 sec-10 min | 15 | sec | 30 | sec | 30 | sec | 15 | sec | 60 | sec | 15 | sec |

According to a Ct value of each group, a relative expression level was calculated according to an equation 2\hat{ }-ΔΔct.

(4) Flow cytometry assay

After culturing was conducted for 96 h, T cells in each group were collected: A cell culture in each well was collected in a 1.5 mL EP tube and centrifuged for 8 min to separate T cells from the cell culture medium, and the supernatant was discarded. Washing was conducted once with PBS, and centrifugation was performed at the same condition to separate cells from PBS. After removing the supernatant, cells were resuspended with 500 μL of phosphate buffered saline (PBS) and fully pipetted up and down by a pipette tip to produce a single-cell suspension. The single-cell suspension was placed on ice for flow cytometry assay.

### (5) Detection of Firefly Luciferase Activity

After culturing was conducted for 72 h and 96 h, T cells in each group were transferred to a 96-well plate for luciferase detection. An equal volume of a Bright-Lumi™ II firefly luciferase reporter gene assay reagent (Shanghai Beyotime Biotech Inc., Item No.: RG052) at room temperature was added, and incubation was conducted at room temperature for 5 min. Chemiluminescence was detected by a multimode microplate reader with a function of measuring chemiluminescence.

### Example 4 Contrast Detection of Various Indexes for Activated T Cells Infected With Mutant Serotypes

### (1) AAV Infection and Culturing of T Cells

Groups: Control AAV6 and viruses 1 to 5 containing mutants of the AAV6 capsid protein

An RPMI 1640 medium was pre-warmed at 37° C. Frozen CD3⁺ T cells were taken and rapidly thawed. Recovered cells were transferred to a 50 mL centrifuge tube, 15 mL of an RPMI 1640 medium including 1% of P/S and 10% of FBS was added to the centrifuge tube, and the centrifuge tube was centrifuged at 300 g for 10 min to 15 min. Cells were resuspended with 1 mL of an RPMI 1640 medium including rhIL-2 at a final concentration of 50 U/mL, a T cell activator (i.e., anti-CD3/CD28 antibodies) at a final concentration of 25 μL/mL, 1% of P/S, and 10% of FBS, and counted (staining was conducted with trypan blue, and a total number of cells and a number of dead cells were counted). According to cell counting results, a cell density was adjusted to 4×10⁵ cells/mL. According to the experimental grouping, 500 μL of a cell suspension (2 × 105 cells/well) was added to a 24-well cell culture plate. T cells in each group were infected at MOI of 1E2, 1E3, 1E4, or 1E5. Infected cells were gently pipetted up and down for thorough mixing, and cultured in an incubator (37° C., 5% CO₂).

### (2) Fluorescence Observation

At 72 h, T cells in each group were subjected to fluorescence imaging by fluorescent microscopy.

(3) Detection of expression levels of target mRNAs

### 1) Total RNA Extraction:

A cell suspension in each group was pipetted to a 1.5 mL centrifuge tube and centrifuged at 300 g for 15 min to collect cells, and a resulting supernatant was discarded. RNA extraction was conducted according to the instructions of TransZol Up Plus RNA Kit (Beijing TransGen Biotech Co., Ltd., Item No.: ER501). 300 μL of TranZol up was added to cells in each tube, and then 60 μL of chloroform was added. Shaking was conducted vigorously for 30 s, and incubation was conducted at room temperature for 3 min. Centrifugation was conducted at 12,000 g and 4° C. for 10 min. A resulting colorless aqueous phase was transferred to a new 1.5 mL RNase-free EP tube, an equal volume of absolute ethanol was added, and thorough mixing was allowed through gentle inverting to produce a solid-liquid mixture. The solid-liquid mixture was loaded on a spin column and centrifuged at 12,000 g and room temperature for 30 s to produce a first filtrate, and the first filtrate was discarded. 500 μL of CB9 was added, centrifugation was conducted at 12,000 g and room temperature for 30 s to produce a second filtrate, and the second filtrate was discarded, which was repeated once. 500 μL of WB9 was added, centrifugation was conducted at 12,000 g and room temperature for 30 s to produce a third filtrate, and the third filtrate was discarded, which was repeated once. Centrifugation was conducted at 12,000 g and room temperature for 2 min to completely remove the residual ethanol. The spin column was placed in a 1.5 mL RNase-free EP tube, 50 μL of RNase-free water was added to a center of the spin column, and standing was allowed at room temperature for 1 min. Centrifugation was conducted at 12,000 g and room temperature for 1 min to elute RNA. A concentration of RNA in a sample was detected by a microvolume nucleic acid quantification instrument. OD260/280 and OD260/230 were determined. The extracted RNA was stored at −80° C. Based on an RNA sample in each group, first-strand cDNA was synthesized with HiScript® III RT SuperMix for qPCR (+gDNA wiper) (Nanjing Vazyme Biotech Co., Ltd., Item No.: R323).

2) qPCR Experiment

With cDNA in each group as a template, a qPCR system was prepared according to the instructions of 2 x SYBR Green qPCR Master Mix (BIMAKE, Item No.: B212203), as shown in Table 3:

TABLE 3
qPCR system

| Reagent | Volume | |
|---|---|---|
| 2×SYBR Green qPCR | 10 | μL |
| Master Mix | | |
| Template | 1 | μL |
| Upstream primer | 0.5 | μL |
| Downstream primer | 0.5 | μL |
| ROX Reference Dye | 0.4 | μL |

| | |
|---|---|
| Deionized water | Up to 20 μL |

Primer sequences (5′->3′) were as follows:

EGFP-Tf:
(SEQ ID NO: 35)
GCTGGAGTACAACTACAAC;
EGFP-Tr:
(SEQ ID NO: 36)
TGGCGGATCTTGAAGTTC;
GAPDH101-F:
(SEQ ID NO: 37)
CTGGGCTACACTGAGCACC;
and
GAPDH101-R:
(SEQ ID NO: 38)
AAGTGGTCGTTGAGGGCAATG.

A qPCR program was set as in Table 4:

TABLE 4
qPCR program

| | 1 | 2 | | | | | | 3 | | | | | |
|---|---|---|---|---|---|---|---|---|---|---|---|---|---|
| Step | Hot-Start DNA Polymerase | PCR | | | | | | Melt Curve | | | | | |
| | Activation | | | | | | | | | | | | |
| | Hold | Cycle (40 cycles) | | | | | | Cycle (1 cycle) | | | | | |
| | | Denature | | Anneal | | Extend | | | | | | | |
| Temp | 95° C. | 95° | C. | 60° | C. | 72° | C. | 95° | C. | 60° | C. | 95° | C. |
| Time | 30 sec-10 min | 15 | sec | 30 | sec | 30 | sec | 15 | sec | 60 | sec | 15 | sec |

According to a Ct value of each group, a relative expression level was calculated according to an equation 2\hat{ }-ΔΔct.

### (4) Flow Cytometry Assay

After culturing was conducted for 72 h, T cells in each group were collected: A cell

culture in each well was collected in a 1.5 mL EP tube and centrifuged for 12 min to produce a first supernatant, and the first supernatant was discarded. Washing was conducted once to produce a second supernatant, and the second supernatant was discarded. Cells were resuspended with 500 μL of PBS and fully pipetted up and down by a pipette tip to produce a single-cell suspension. The single-cell suspension was placed on ice for flow cytometry assay.

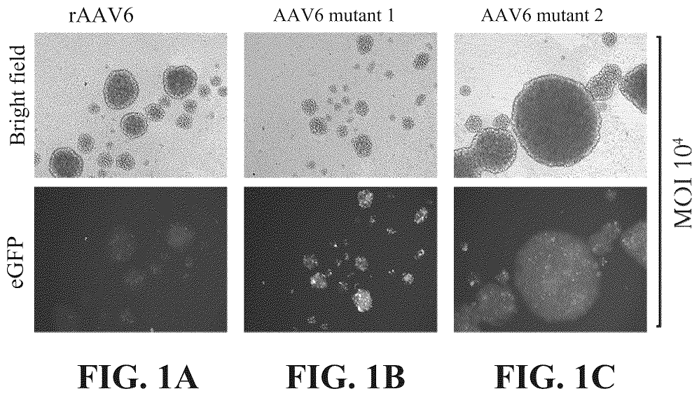

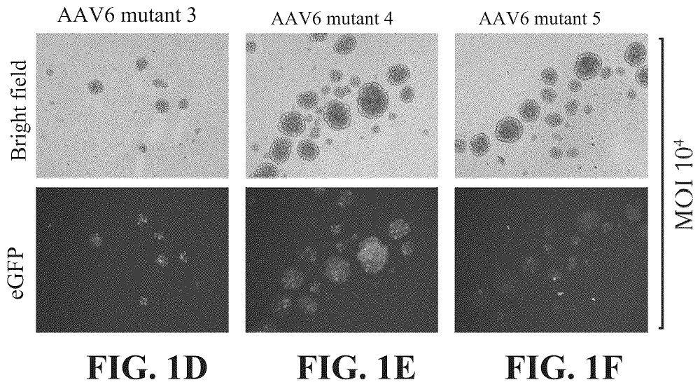

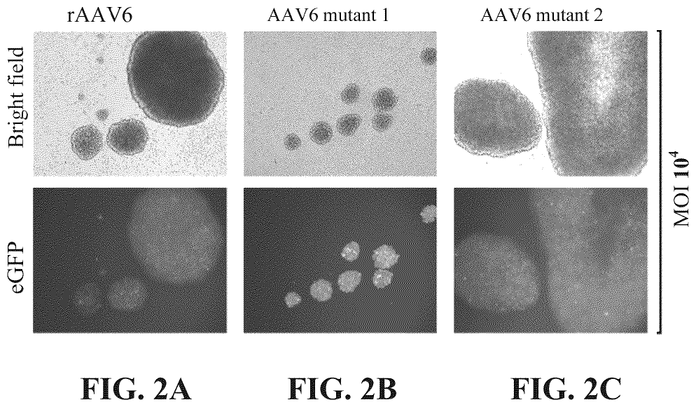

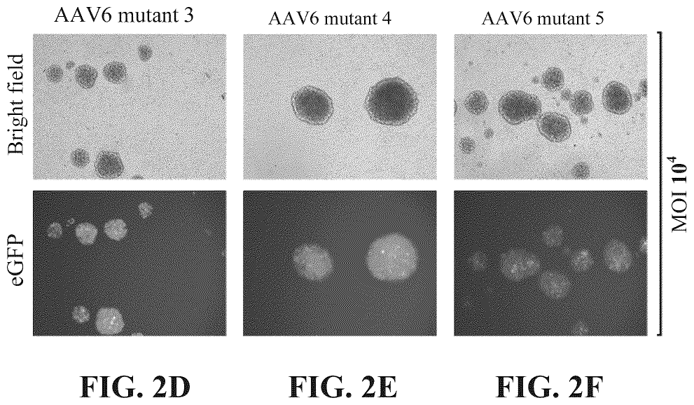

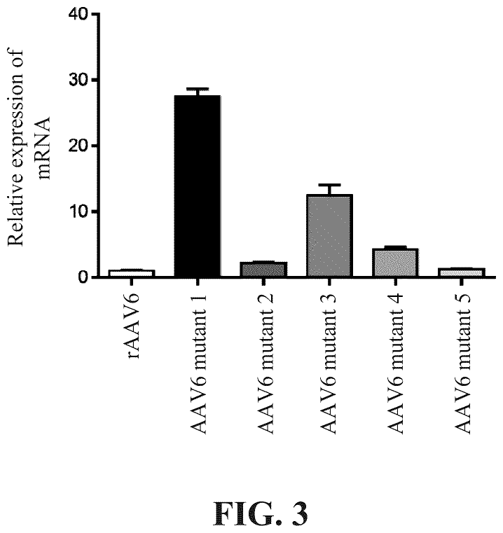

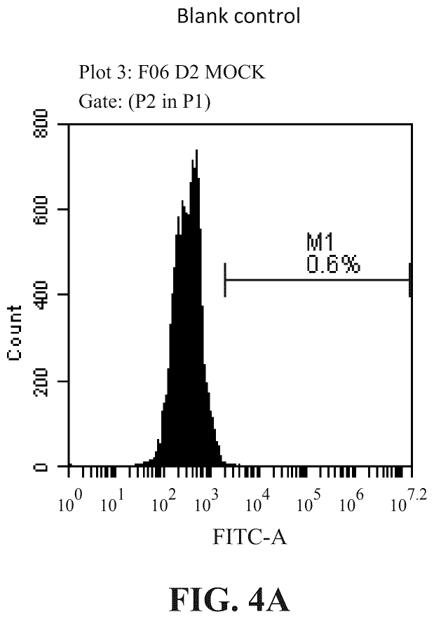

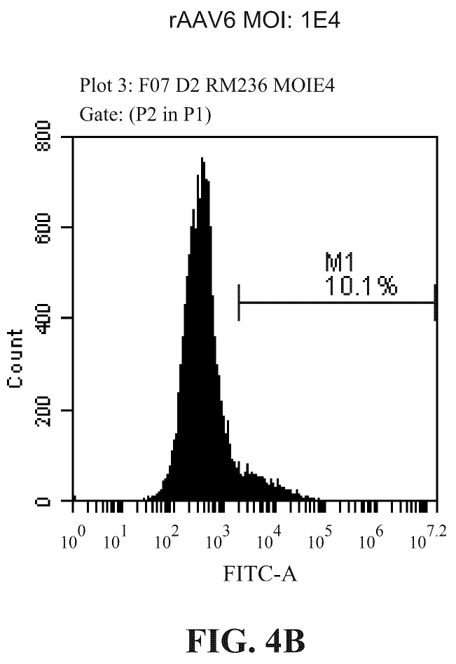

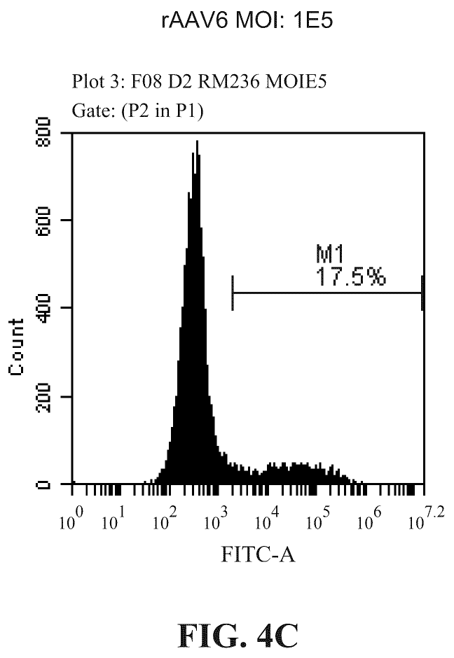

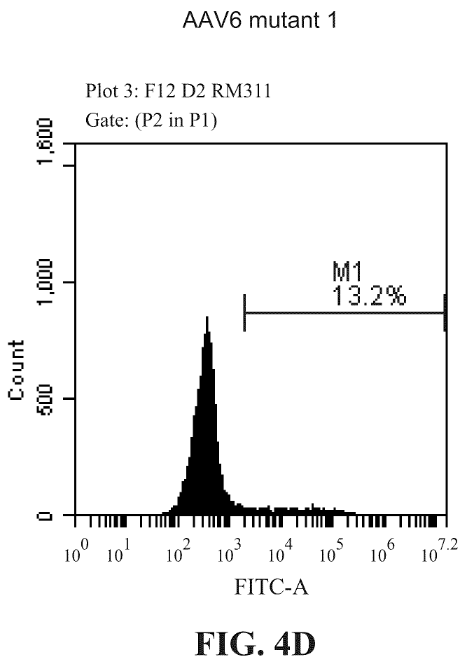

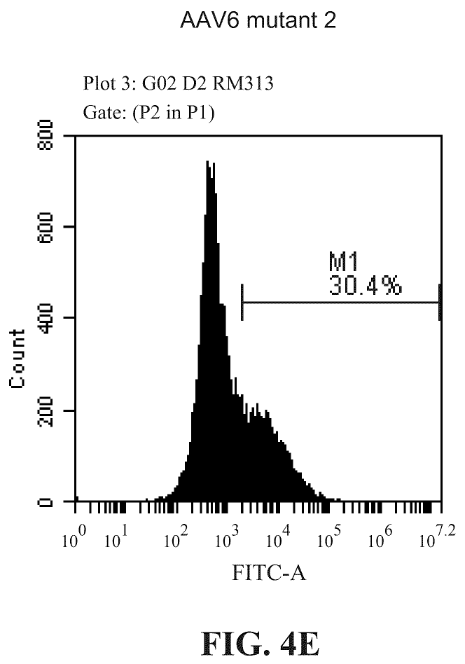

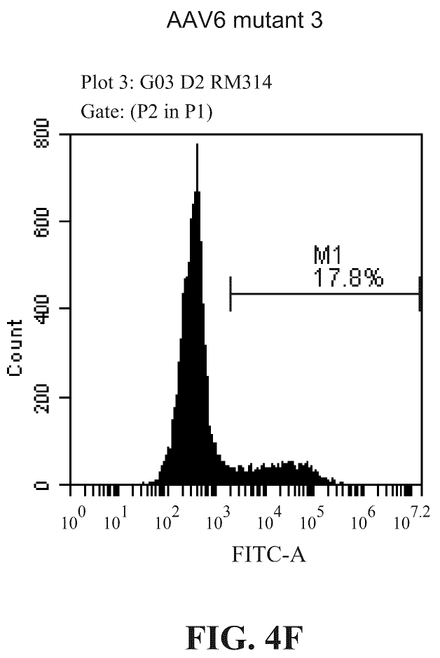

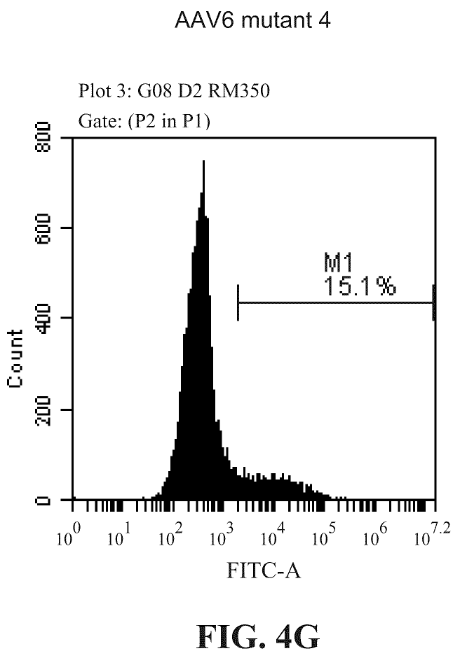

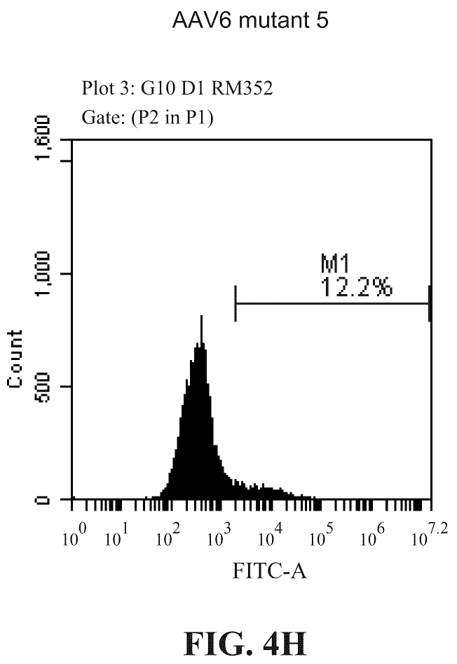

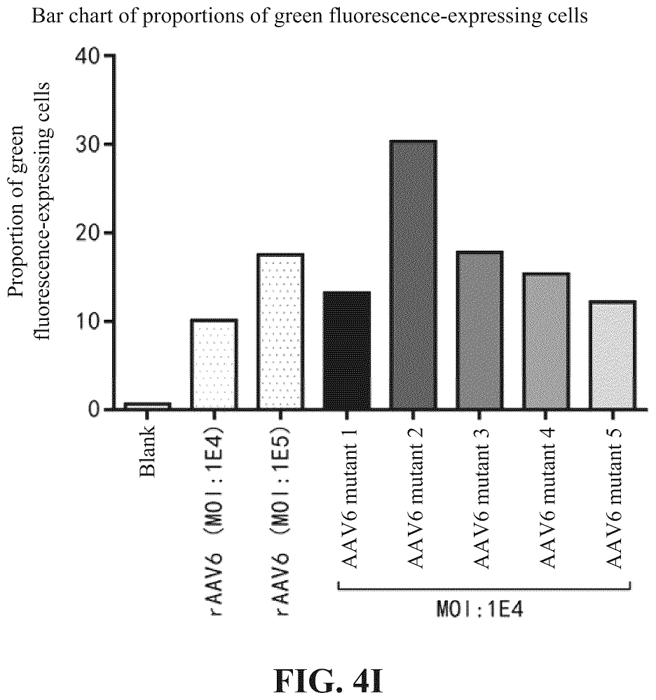

Results of the infection of resting T cells with viruses 1 to 5 containing mutants of the AAV capsid protein showed that viruses containing the capsid protein mutants exhibited a higher eGFP fluorescence intensity than the control rAAV6 at different time points (72 h and 96 h). The virus 1 containing the capsid protein mutant had the most significant difference from the control rAAV6 (FIG. 1A to FIG. 1F and FIG. 2A to FIG. 2F). RT-qPCR results showed that, at the same MOI, viruses 1 to 5 containing the capsid protein mutants enabled a 28-fold to 1.5-fold higher expression level of mRNA of a target gene than the control rAAV6 (FIG. 3). Flow cytometry analysis showed that, compared with the control rAAV6, viruses 1 to 5 containing the capsid protein mutants all made a proportion of eGFP-positive cells improved (FIG. 4A to FIG. 4I). The viruses 2 and 3 containing the capsid protein mutants at MOI of 1E4 achieved the same proportion of infected cells (expressing eGFP fluorescence) among resting T cells as the control rAAV6 at MOI of 1E5. In particular, when resting T cells were infected by virus 2 containing the capsid protein mutant at MOI of 1E4, 30% of the resting T cells expressed eGFP. In contrast, when resting T cells were infected with the control rAAV6 at MOI of 1E5, only 17.5% of the resting T cells expressed green fluorescence, which was merely improved by 7.5% compared with a proportion of green fluorescence-expressing cells among resting T cells infected with the control rAAV6 at MOI of 1E4.

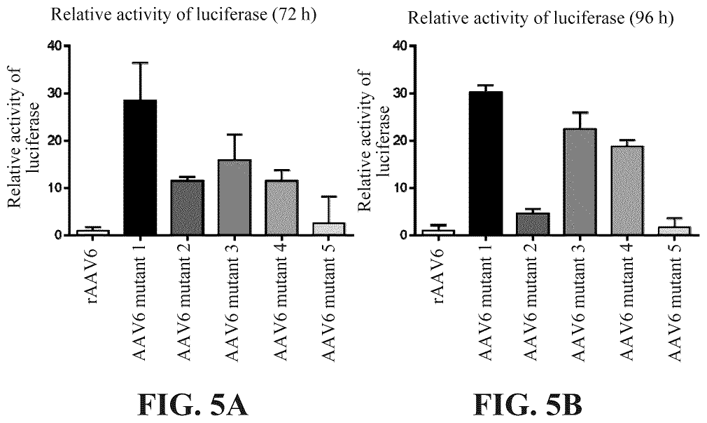

Results of luciferase activity assay for lysates of infected T cells showed that, at 72 h and 96 h after infection, viruses 1 to 5 containing the capsid protein mutant all led to a higher luciferase activity in T cells than the control rAAV6 (FIG. 5A and FIG. 5B). It indicates that the mutants of the AAV capsid protein of the present application has a prominent infection effect for resting T cells.

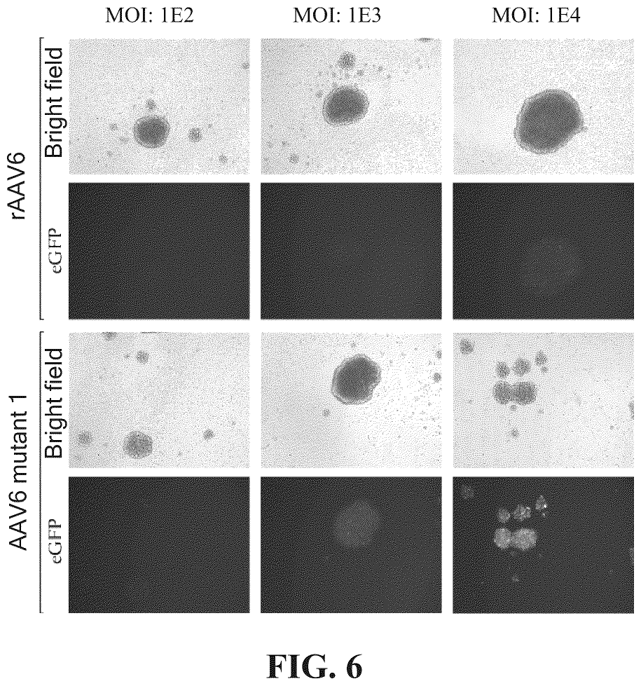

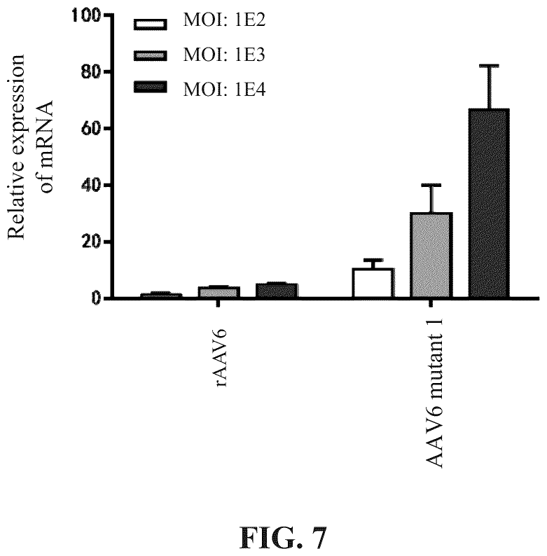

According to the experiment to compare viral infectivity at different MOIs, when used for the infection at MOI of 1E2, 1E3, and 1E4, the virus 1 containing the mutant of the AAV capsid protein led to a significantly higher eGFP fluorescence intensity and relative mRNA expression level than the control rAAV6 (FIG. 6 and FIG. 7). It indicates that the mutants of the AAV capsid protein of the present application has the potential to achieve high efficacy at a low dose, which can reduce the immune response elicited by rAAV in a therapy.

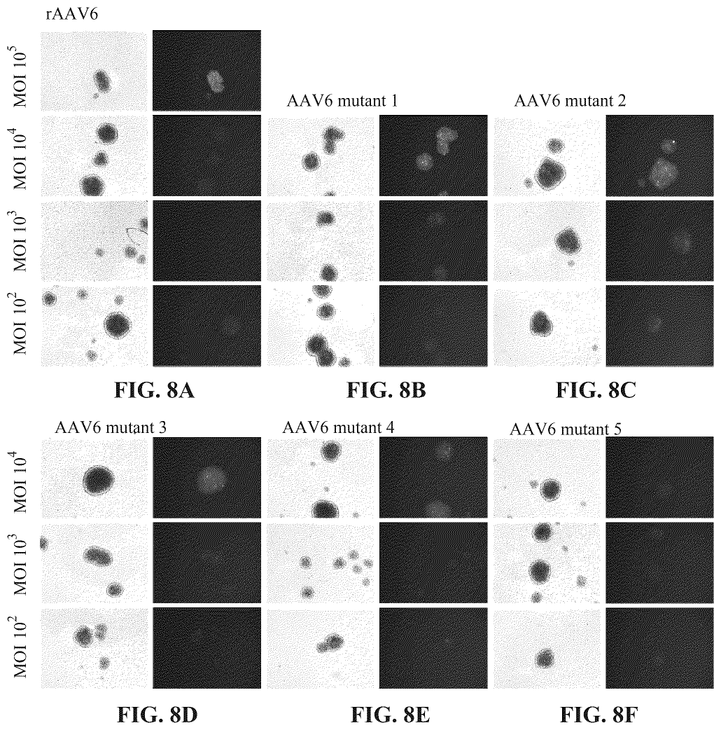

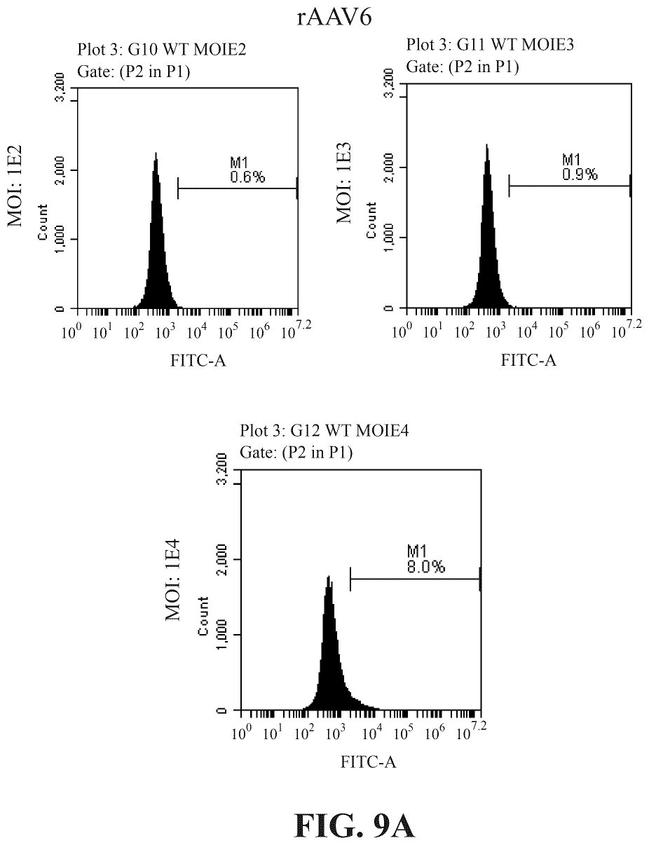

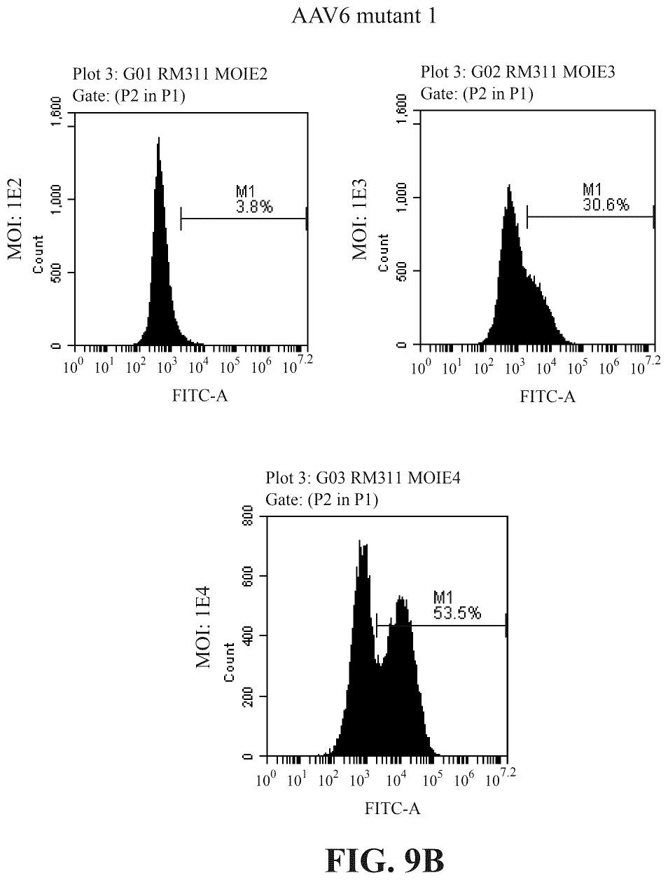

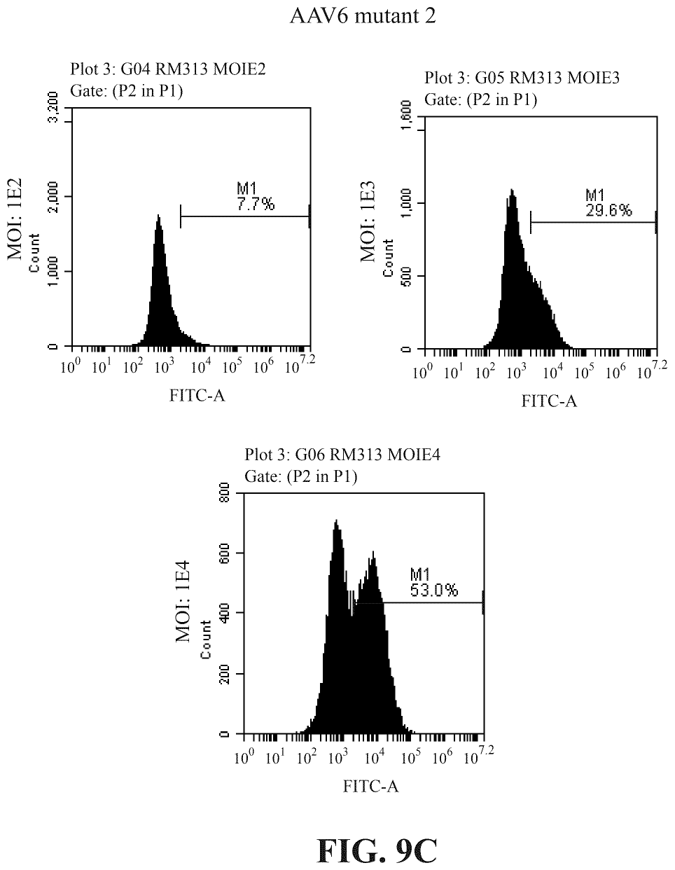

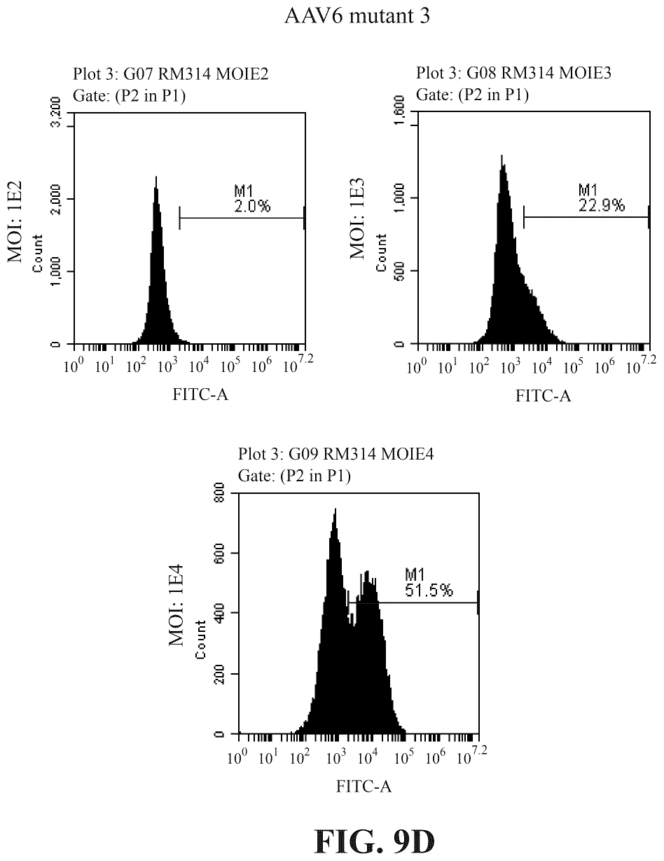

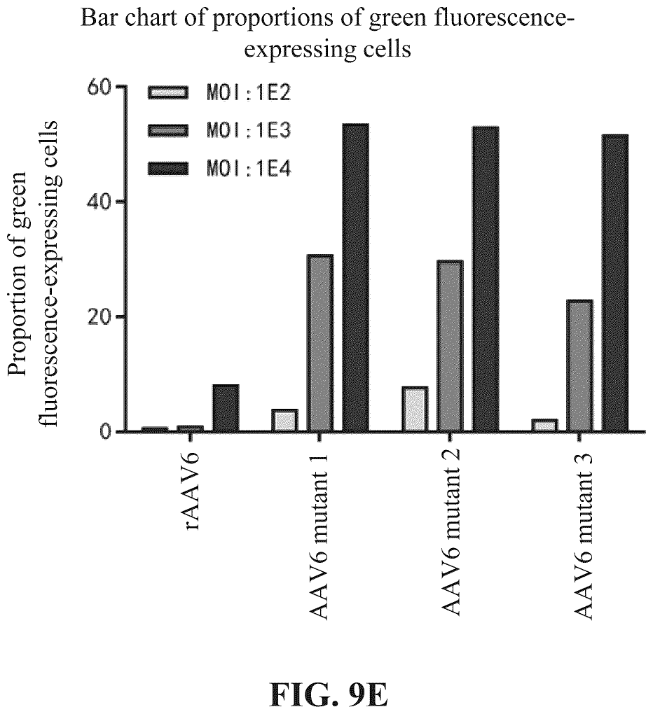

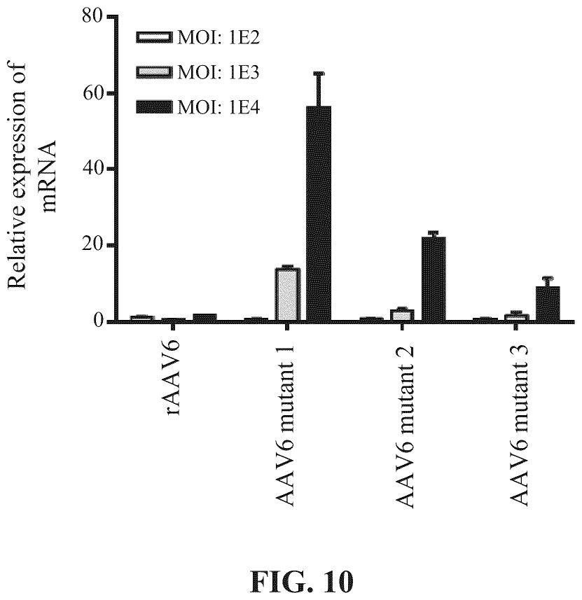

Moreover, viruses 1 to 3 containing mutants of the AAV capsid protein performed exceptionally well when infecting activated T cells. Flow cytometry assay results revealed that, at MOI of 1E2, the control rAAV6 exhibited very weak infectivity for activated T cells, and less than 1% of the cells expressed eGFP (as shown in FIG. 8A to FIG. 8F). When viruses 1 to 3 containing mutants of the AAV capsid protein infected activated T cells at MOI of 1E2, proportions of eGFP-expressing cells were 3.8%, 7.7%, and 2.0%, respectively. When the control rAAV6 infected activated T cells at MOI increasing 10-fold to 1E3, a proportion of eGFP-expressing cells was still less than 1%. When viruses 1 to 3 containing mutants of the AAV capsid protein infected activated T cells at MOI of 1E3, proportions of eGFP-expressing cells reached 20% to 30%. When viruses 1 to 3 containing mutants of the AAV capsid protein infected activated T cells at MOI of 1E4, proportions of eGFP-expressing cells all were 50% or more. However, when the control rAAV6 infected activated T cells at MOI of 1E4, a proportion of eGFP-expressing cells was merely 8% (FIG. 9A to FIG. 9E). RT-qPCR results were consistent with the results of immunofluorescence observation and flow cytometry analysis. When infecting activated T cells at MOI of 1E4, viruses 1 to 3 containing the capsid protein mutant enabled a 6-fold to 36-fold higher expression level of mRNA of a target gene than the control rAAV6 (FIG. 10).

The rAAV mutant constructed from the mutant of the AAV capsid protein in the present application can efficiently target resting or activated T cells, and has advantages such as low dose, strong infectivity, and high safety. Compared with the infection (48 h) after stimulation with a stimulating factor in the prior art, the infection of inactivated T cells can allow the collection, infection, and reinfusion of T cells on the same day, which can greatly reduce the preparation cost. Moreover, the inactivated T cells reduce the in vitro culturing and processing time, which can reduce the influence on the activity of T cells. The infection by lentiviruses involves the genomic integration and has a relatively low infection efficiency for inactivated T cells. The mutant of the AAV capsid protein in the present application does not need to be integrated into a genome, which enables the quick infection and reinfusion and reduces the unnecessary quality control and in vitro dwell time. The rAAV mutant constructed from the mutant of the AAV capsid protein in the present application also exhibits very excellent infectivity for T cells stimulated with a stimulating factor. In summary, the infection of resting or activated T cells with the rAAV mutant carrying the mutant of the AAV capsid protein of the present application has great clinical values and commercial application prospects.

Finally, it should be noted that the above examples are provided merely to describe the technical solutions of the present application, rather than to limit the protection scope of the present application. Although the present application is described in detail with reference to preferred examples, those of ordinary skill in the art should understand that modifications or equivalent replacements may be made to the technical solutions of the present application without departing from the spirit and scope of the technical solutions of the present application.

1. A heterologous peptide targeting a T cell, wherein an amino acid sequence of the heterologous peptide is set forth in any one of SEQ ID NOS: 1-5.

2. The heterologous peptide according to claim 1, wherein a nucleotide sequence encoding the heterologous peptide is set forth in any one of SEQ ID NOS: 6-10.

3. A mutant of an adeno-associated virus (AAV) capsid protein targeting a T cell, comprising the heterologous peptide according to claim 1.

4. The mutant of the AAV capsid protein according to claim 3, wherein the mutant of the AAV capsid protein is produced by inserting the heterologous peptide into an AAV capsid protein or substituting 5 to 20 amino acids of the AAV capsid protein with the heterologous peptide.

5. The mutant of the AAV capsid protein according to claim 4, wherein an insertion site for the heterologous peptide is located between amino acids 588 and 589 of the AAV capsid protein.

6. The mutant of the AAV capsid protein according to claim 5, wherein an amino acid sequence of the mutant of the AAV capsid protein is set forth in any one of SEQ ID NOS: 11-15.

7. The mutant of the AAV capsid protein according to claim 6, wherein a nucleotide sequence encoding the mutant of the AAV capsid protein is set forth in any one of SEQ ID NOS: 16-20.

8. A recombinant adeno-associated virus (rAAV) targeting a T cell, comprising the mutant of the AAV capsid protein according to claim 3.

9. A rAAV targeting a T cell, comprising the mutant of the AAV capsid protein according to claim 6.

10. The rAAV according to claim 8, further comprising a heterologous target gene.

11. The rAAV according to claim 10, wherein the heterologous target gene encodes any gene product selected from the group consisting of interference RNA, an aptamer, an endonuclease, and a guide RNA.

12. A pharmaceutical composition for delivering a gene product to a cell of a subject, comprising the heterologous peptide according to claim 1, a mutant of an AAV capsid protein comprising the heterologous peptide according to claim 1, or a rAAV comprising a mutant of an AAV capsid protein comprising the heterologous peptide according to claim 1.

13. The pharmaceutical composition according to claim 12, wherein the cell is an immune cell.

14. A method for infecting a resting or activated T cell, comprising allowing the rAAV according to claim 8 to contact the resting or activated T cell.

15. A method for infecting a resting or activated T cell, comprising allowing the rAAV according to claim 10 to contact the resting or activated T cell.

16. A pharmaceutical composition for tumor immunotherapy, comprising the heterologous peptide according to claim 1, a mutant of an AAV capsid protein comprising the heterologous peptide according to claim 1, or a therapeutically effective amount of a rAAV comprising a mutant of an AAV capsid protein comprising the heterologous peptide according to claim 1.

17. The pharmaceutical composition for tumor immunotherapy according to claim 16, wherein the tumor immunotherapy comprises a CAR-T therapy or a TCR-T therapy.
